(19) United States
(12) Patent Application Publication
(10) Pub. No.: US 2007/0173536 A1
(43) Pub. Date: Jul. 26, 2007

(54) Crystalline forms of zolmitriptan

(75) Inventors: Paul Adriaan Van Der Schaaf, Hagenthal-le-Haut (FR); Fritz Blatter, Reinach (CH); Martin Szelagiewicz, Munchenstein (CH); Ulrich Berens, Binzen (DE); Susan De Paul, Zurich (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(73) Assignee: CIBA SPECIALTY CHEMICALS HOLDING INC., Basel (CH)

(21) Appl. No.: 10/588,176

(22) PCT Filed: Jan. 28, 2005
(86) PCT No.: PCT/EP05/50362

§ 371 Date: Aug. 2, 2006

Related U.S. Application Data
(60) Provisional application No. 60/543,107, filed on Feb. 9, 2004.

(30) Foreign Application Priority Data
Feb. 6, 2004 (EP) 04100452.4

(51) Int. Cl. A61K 31/422; C07D 413/02

(52) U.S. Cl. 514/376; 548/229

(57) ABSTRACT

The present invention is directed to a novel crystalline form of Zolmitriptan, herein designated as Form A, and several novel solvates of Zolmitriptan, herein designated as Form B, C, D, E, F, and G, processes for the preparation thereof and pharmaceutical compositions comprising these crystalline forms.

The present invention is directed to crystalline forms of Zolmitriptan, processes for the preparation thereof and pharmaceutical compositions comprising these crystalline forms.

The present invention relates to crystalline forms of Zolmitriptan. Zolmitriptan is known by the chemical name, (S)-4-[[3-[2-(dimethylamino)ethyl]-1H-indol-5-yl]methyl]-2-oxazolidinone. Zolmitriptan has the following formula:

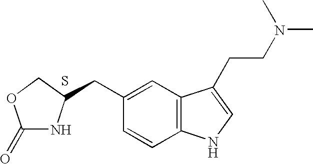

Zolmitriptan is a selective 5-hydroxytryptamine_{1B/1D}(5-HT_{1B/1D}) receptor agonist. Zolmitriptan is marketed as an oral formulation for acute treatment of migraine.

Processes for the preparation of Zolmitriptan are described in WO-A-91/18897, WO-A-97/06162, and in the publications by J. Buckingham et al. in Journal of Medicinal Chemistry (1995), vol. 38, pages 3566-3580 and J. Ngo et al. in Drugs of the Future (1997), vol. 22, pages 260-269. The processes in the above mentioned patents and publications results either in the preparation of Zolmitriptan isopropanolate containing half a molcule of water, or a Zolmitriptan ethyl acetate solvate which is subsequently treated in an acetone/water mixture. These patents and publications do not describe the crystallinity of the products or the characterization of the solid state properties. It is known that pharmaceutical substances can exhibit polymorphism. Polymorphism is commonly defined as the ability of any substance to have two or more different crystal structures. Drug substances may also encapsulate solvent molecules when crystallized. These solvates or hydrates are referred to as pseudopolymorphs. Solvated forms may contain substantial amounts of residual solvent and are normally not practical for the manufacturing of pharmaceutical products. However, such solvates may be valuable intermediates to prepare stable, and solvent free crystal forms. It is therefore important to find appropriate processes to transform solvated forms into non-solvated forms by suitable desolvation processes. Different polymorphs, pseudopolymorphs or the amorphous form differ in their physical properties such as melting point, solubility etc. These can appreciably influence pharmaceutical properties such as dissolution rate and bioavailability. It is therefore important to evaluate polymorphism of drug substances. Furthermore, the discovery of new crystalline polymorphic forms of a drug enlarge the repertoire of materials that a formulation scientist has with which to design a pharmaceutical dosage form of a drug with a targeted release profile or other desired characteristics. We now have surprisingly found Zolmitriptan to be polymorph and disclose a novel crystalline form of Zolmitriptan, herein designated as From A, and several novel solvates of Zolmitriptan, herein designated as Form B, C, D, E, F, and G.

Accordingly, the present invention is directed to the polymorphic and pseudopolymorphic Forms A, B, C, D, E, F and G of Zolmitriptan and processes for preparing them.

One object of the invention is a crystalline form of (S)-4-[[3-[2-(dimethylamino)ethyl]-1H-indol-5-yl]methyl]-2-oxazolidinone, herein designated as Form A, which exhibits a characteristic X-ray powder diffraction pattern with characteristic peaks expressed in d-values (Å) and in 20 as given in Table 1. Intensities are expressed hereinafter using the following abbreviations: vs=very strong intensity, s=strong intensity, m=medium intensity, w=weak intensity, vw=very weak intensity.

TABLE 1
d-spacings and 2θ angles for Form A.

| Angle °2θ | d-spacing (Å) | Qualitative Relative Intensity |
|---|---|---|
| 9.8 | 9.0 | vw |
| 11.5 | 7.7 | w |
| 12.1 | 7.3 | w |
| 12.5 | 7.1 | m |
| 13.9 | 6.4 | s |
| 14.4 | 6.15 | s |
| 15.5 | 5.69 | s |
| 16.6 | 5.32 | vw |
| 17.4 | 5.10 | w |
| 18.3 | 4.83 | vw |
| 19.3 | 4.59 | vs |
| 19.6 | 4.53 | s |
| 20.7 | 4.29 | w |
| 21.1 | 4.22 | m |
| 22.1 | 4.02 | s |
| 23.1 | 3.85 | m |
| 24.0 | 3.71 | vs |
| 24.3 | 3.66 | shoulder |
| 25.4 | 3.51 | w |
| 25.8 | 3.45 | m |
| 27.4 | 3.25 | m |
| 28.1 | 3.17 | w |
| 29.0 | 3.08 | s |
| 30.6 | 2.92 | w |
| 31.2 | 2.86 | m |
| 33.0 | 2.71 | w |
| 34.4 | 2.61 | w |
| 35.8 | 2.50 | m |

Besides by the X-ray powder diffraction pattern depicted in FIG. 1, Form A is distinguishably characterised by characteristic peaks expressed in d-values (Å) at 6.4 (s), 6.15 (s), 5.69 (s), 4.59 (vs), 4.53 (s), 4.02 (s), 3.71 (vs), 3.08 (s); more preferably by characteristic peaks expressed in d-values (Å) at 7.1 (m), 6.4 (s), 6.15 (s), 5.69 (s), 4.59 (vs), 4.53 (s), 4.22 (m), 4.02 (s), 3.85 (m), 3.71 (vs), 3.45 (m), 3.25 (m), 3.08 (s), 2.86 (m), 2.50 (m); most preferably by characteristic peaks expressed in d-values (Å) at 7.7 (w), 7.3 8w), 7.1 (m), 6.4 (s), 6.15 (s), 5.69 (s), 5.10 (w), 4.59 (vs), 4.53 (s), 4.29 (w), 4.22 (m), 4.02 (s), 3.85 (m), 3.71 (vs), 3.51 (w), 3.17 (w), 3.45 (m), 3.25 (m), 3.08 (s), 2.92 (w), 2.86 (m), 2.71 (w), 2.61 (w), 2.50 (m).

Another object of the invention is a crystalline 1-butanol solvate of (S)-4-[[3-[2-(dimethylamino)ethyl]-1H-indol-5-yl]methyl]-2-oxazolidinone, herein designated as Form B, which exhibits a characteristic X-ray powder diffraction pattern with characteristic peaks expressed in d-values (Å) and in 2θ as given in Table 2.

TABLE 2
d-spacings and 2θ angles for Form B.

| Angle °2θ | d-spacing (Å) | Qualitative Relative Intensity |
|---|---|---|
| 8.3 | 10.7 | m |
| 8.8 | 10.0 | w |
| 11.3 | 7.9 | vw |
| 11.8 | 7.5 | s |
| 13.0 | 6.8 | w |
| 13.9 | 6.4 | m |
| 14.6 | 6.05 | m |
| 15.7 | 5.64 | w |
| 16.5 | 5.36 | m |
| 17.2 | 5.16 | m |
| 17.7 | 5.01 | m |
| 18.2 | 4.87 | s |
| 18.5 | 4.78 | w |
| 19.2 | 4.61 | m |
| 19.8 | 4.48 | s |
| 21.9 | 4.05 | s |
| 22.5 | 3.94 | w |
| 22.8 | 3.90 | m |
| 23.4 | 3.80 | w |
| 23.6 | 3.76 | s |
| 24.1 | 3.69 | m |
| 25.3 | 3.52 | m |
| 26.6 | 3.34 | w |
| 27.2 | 3.28 | vw |
| 27.5 | 3.24 | w |
| 29.4 | 3.03 | w |
| 29.7 | 3.01 | w |
| 30.5 | 2.93 | w |
| 31.7 | 2.82 | w |

Besides by the X-ray powder diffraction pattern depicted in FIG. 2, Form B is distinguishably characterised by characteristic peaks expressed in d-values (Å) at 7.5 (s), 4.87 (s), 4.48 (s), 4.05 (s), 3.76 (s); more preferably by characteristic peaks expressed in d-values (Å) at 10.7 (m), 7.5 (s), 6.4 (m), 6.05 (m), 5.36 (m), 5.16 (m), 5.01 (m), 4.87 (s), 4.61 (m), 4.48 (s), 4.05 (s), 3.90 (m), 3.76 (s), 3.69 (m), 3.52 (m); most preferably by characteristic peaks expressed in d-values (Å) at 10.7 (m), 10.0 (w), 7.5 (s), 6.8 (w), 6.4 (m), 6.05 (m), 5.64 (w), 5.36 (m), 5.16 (m), 5.01 (m), 4.87 (s), 4.78 (w), 4.61 (m), 4.48 (s), 4.05 (s), 3.94 (w), 3.90 (m), 3.80 (w), 3.76 (s), 3.69 (m), 3.52 (m), 3.34 (w), 3.24 (w), 3.03 (w), 3.01 (w), 2.93 (w), 2.82 (w).

Another object of the invention is a crystalline anisol solvate of (S)-4-[[3-[2-(dimethylamino)ethyl]-1H-indol-5-yl]methyl]-2-oxazolidinone, herein designated as Form C, which exhibits a characteristic X-ray powder diffraction pattern with characteristic peaks expressed in d-values (Å) and in 2θ as given in Table 3.

TABLE 3
d-spacings and 2θ angles for Form C.

| Angle °2θ | d-spacing (Å) | Qualitative Relative Intensity |
|---|---|---|
| 8.5 | 10.4 | m |
| 9.0 | 9.8 | vw |
| 11.4 | 7.8 | s |
| 12.7 | 7.0 | vw |
| 13.4 | 6.6 | m |
| 13.8 | 6.4 | s |
| 14.3 | 6.18 | m |
| 15.0 | 5.89 | m |
| 15.6 | 5.68 | w |
| 17.0 | 5.23 | w, shoulder |
| 17.1 | 5.17 | m |
| 17.7 | 5.00 | m |
| 18.1 | 4.89 | s |
| 18.3 | 4.84 | w, shoulder |
| 18.6 | 4.77 | vw |
| 20.0 | 4.44 | vs |
| 21.0 | 4.23 | w |
| 21.5 | 4.13 | vw |
| 22.2 | 4.00 | s |
| 22.7 | 3.92 | m |
| 23.4 | 3.80 | m |
| 24.0 | 3.70 | vs |
| 24.4 | 3.65 | vw |
| 25.1 | 3.54 | m |
| 25.7 | 3.46 | s |
| 26.1 | 3.41 | m |
| 27.6 | 3.23 | w |
| 28.3 | 3.15 | w |
| 28.9 | 3.09 | w |
| 30.3 | 2.95 | w |
| 30.6 | 2.92 | w |
| 31.5 | 2.84 | vw |
| 32.1 | 2.78 | vw |
| 33.3 | 2.69 | w |
| 33.8 | 2.65 | w |

Besides by the X-ray powder diffraction pattern depicted in FIG. 3, Form C is distinguishably characterised by peaks expressed in d-values (Å) at 7.8 (s), 6.4 (s), 4.89 (s), 4.44 (vs), 4.00 (s), 3.70 (vs), 3.46 (s); more preferably at 10.4 (m), 7.8 (s), 6.6 (m), 6.4 (s), 6.18 (m), 5.89 (m), 5.17 (m), 5.00 (m), 4.89 (s), 4.44 (vs), 4.00 (s), 3.92 (m), 3.80 (m), 3.70 (vs), 3.54 (m), 3.46 (s), 3.41 (m); most preferably at 10.4 (m), 7.8 (s), 6.6 (m), 6.4 (s), 6.18 (m), 5.89 (m), 5.68 (w), 5.23 (w, shoulder), 5.17 (m), 5.00 (m), 4.89 (s), 4.84 (w, shoulder), 4.44 (vs), 4.23 (w), 4.00 (s), 3.92 (m), 3.80 (m), 3.70 (vs), 3.54 (m), 3.46 (s), 3.41 (m), 3.23 (w), 3.15 (w), 3.09 (w), 2.95 (w), 2.92 (w), 2.69 (w), 2.65 (w).

Another object of the invention is a crystalline 2-propanol solvate of (S)-4-[[3-[2-(dimethylamino)ethyl]-1H-indol-5-yl]methyl]-2-oxazolidinone, herein designated as Form D, which exhibits a characteristic X-ray powder diffraction pattern with characteristic peaks expressed in d-values (Å) and in 2θ as given in Table 4.

TABLE 4
d-spacings and 2θ angles for Form D.

| Angle °2θ | d-spacing (Å) | Qualitative Relative Intensity |
|---|---|---|
| 8.2 | 10.7 | s |
| 8.8 | 10.0 | m |
| 11.6 | 7.6 | vs |
| 12.8 | 6.9 | m |
| 13.6 | 6.5 | vw |
| 14.0 | 6.3 | s |
| 14.6 | 6.1 | m |
| 15.5 | 5.71 | w |
| 16.5 | 5.38 | vw |
| 17.0 | 5.21 | s |
| 17.6 | 5.03 | s |
| 18.2 | 4.86 | vs |
| 18.5 | 4.80 | shoulder |
| 19.2 | 4.62 | m |
| 19.7 | 4.50 | vs |
| 21.6 | 4.11 | s |
| 21.8 | 4.07 | m |
| 22.8 | 3.90 | s |
| 23.3 | 3.81 | m |
| 23.7 | 3.75 | m |
| 24.1 | 3.69 | s |
| 25.3 | 3.52 | s |
| 25.6 | 3.47 | vw |
| 26.2 | 3.40 | m |
| 27.2 | 3.27 | w |
| 27.5 | 3.24 | w |
| 29.5 | 3.02 | w |
| 30.5 | 2.93 | w |
| 31.6 | 2.83 | w |
| 32.9 | 2.72 | vw |

Besides by the X-ray powder diffraction pattern depicted in FIG. 4, Form D is distinguishably characterised by peaks expressed in d-values (Å) at 10.7 (s), 7.6 (vs), 6.3 (s), 5.21 (s), 5.03 (s), 4.86 (vs), 4.50 (vs), 4.11 (s), 3.90 (s), 3.69 (s), 3.52 (s); more preferably at 10.7 (s), 10.0 (m), 7.6 (vs), 6.9 (m), 6.3 (s), 6.1 (m), 5.21 (s), 5.03 (s), 4.86 (vs), 4.62 (m), 4.50 (vs), 4.11 (s), 4.07 (m), 3.90 (s), 3.81 (m), 3.75 (m), 3.69 (s), 3.52 (s), 3.40 (m); most preferably at 10.7 (s), 10.0 (m), 7.6 (vs), 6.9 (m), 6.3 (s), 6.1 (m), 5.71 (w), 5.21 (s), 5.03 (s), 4.86 (vs), 4.62 (m), 4.50 (vs), 4.11 (s), 4.07 (m), 3.90 (s), 3.81 (m), 3.75 (m), 3.69 (s), 3.52 (s), 3.40 (m), 3.27 (w), 3.24 (w), 3.02 (w), 2.93 (w), 2.83 (w).

Another object of the invention is a crystalline ethyl methyl ketone solvate of (S)-4-[[3-[2-(dimethylamino)ethyl]-1H-indol-5-yl]methyl]-2-oxazolidinone, herein designated as Form E, which exhibits a characteristic X-ray powder diffraction pattern with characteristic peaks expressed in d-values (Å) and in 2θ as given in Table 5.

TABLE 5
d-spacings and 2θ angles for Form E.

| Angle °2θ | d-spacing (Å) | Qualitative Relative Intensity |
|---|---|---|
| 8.1 | 10.9 | m |
| 8.8 | 10.1 | w |
| 11.1 | 8.0 | w |
| 12.1 | 7.3 | vs |
| 13.7 | 6.5 | m |
| 14.3 | 6.2 | s |
| 15.8 | 5.60 | vw |
| 17.2 | 5.10 | m |
| 17.5 | 5.06 | m |
| 17.7 | 5.00 | shoulder |
| 18.3 | 4.85 | s |
| 18.7 | 4.75 | m |
| 19.0 | 4.66 | s |
| 19.8 | 4.47 | vs |
| 22.0 | 4.03 | s |
| 22.3 | 3.98 | s |
| 22.7 | 3.92 | shoulder |
| 23.4 | 3.80 | m |
| 23.9 | 3.72 | s |
| 24.2 | 3.67 | m |
| 24.4 | 3.64 | m |
| 25.0 | 3.55 | s |
| 25.7 | 3.46 | w |
| 26.4 | 3.37 | w |
| 26.9 | 3.31 | m |
| 29.4 | 3.03 | w |
| 31.0 | 2.88 | w |
| 31.9 | 2.80 | w |

Besides by the X-ray powder diffraction pattern depicted in FIG. 5, Form E is distinguishably characterised by peaks expressed in d-values (Å) at 7.3 (vs), 6.2 (s), 4.85 (s), 4.66 (s), 4.47 (vs), 4.03 (s), 3.98 (s), 3.72 (s), 3.55 (s); more preferably at 10.9 (m), 7.3 (vs), 6.5 (m), 6.2 (s), 5.10 (m), 5.06 (m), 4.85 (s), 4.75 (m), 4.66 (s), 4.47 (vs), 4.03 (s), 3.98 (s), 3.80 (m), 3.72 (s), 3.67 (m), 3.64 (m), 3.55 (s), 3.31 (m); most preferably at 10.9 (m), 10.1 (w), 8.0 (w), 7.3 (vs), 6.5 (m), 6.2 (s), 5.10 (m), 5.06 (m), 4.85 (s), 4.75 (m), 4.66 (s), 4.47 (vs), 4.03 (s), 3.98 (s), 3.80 (m), 3.72 (s), 3.67 (m), 3.64 (m), 3.55 (s), 3.46 (w), 3.37 (w), 3.31 (m), 3.03 (w), 2.88 (w), 2.80 (w).

Another object of the invention is a crystalline tetrahydrofuran solvate of (S)-4-[[3-[2-(dimethylamino)ethyl]-1H-indol-5-yl]methyl]-2-oxazolidinone, herein designated as Form F, which exhibits a characteristic X-ray powder diffraction pattern with characteristic peaks expressed in d-values (Å) and in 2θ as given in Table 6.

TABLE 6
d-spacings and 2θ angles for Form F.

| Angle °2θ | d-spacing (Å) | Qualitative Relative Intensity |
|---|---|---|
| 8.4 | 10.5 | m |
| 8.8 | 10.0 | w |
| 11.7 | 7.6 | s |
| 12.9 | 6.8 | m |
| 13.6 | 6.5 | vw |
| 14.2 | 6.2 | m |
| 14.8 | 5.97 | s |
| 15.7 | 5.65 | w |
| 16.8 | 5.28 | m |
| 17.1 | 5.18 | m |
| 17.6 | 5.02 | shoulder |
| 17.8 | 4.98 | s |
| 18.3 | 4.84 | s |
| 18.6 | 4.78 | shoulder |
| 19.5 | 4.55 | m |
| 19.9 | 4.46 | m |
| 21.6 | 4.11 | vs |
| 21.9 | 4.05 | m |
| 22.7 | 3.92 | vw |
| 23.1 | 3.85 | m |
| 23.3 | 3.82 | m |
| 23.9 | 3.72 | vs |
| 24.3 | 3.66 | vs |
| 25.2 | 3.53 | vw |
| 25.5 | 3.50 | m |
| 25.8 | 3.45 | w |
| 26.2 | 3.40 | m |
| 26.7 | 3.34 | vw |
| 27.4 | 3.25 | m |
| 28.5 | 3.13 | w |
| 29.9 | 2.99 | vw |
| 31.8 | 2.81 | w |

Besides by the X-ray powder diffraction pattern depicted in FIG. 6, Form F is distinguishably characterised by peaks expressed in d-values (Å) at 7.6 (s), 5.97 (s), 4.98 (s), 4.84 (s), 4.11 (vs), 3.72 (vs), 3.66 (vs); more preferably at 10.5 (m), 7.6 (s), 6.8 (m), 6.2 (m), 5.97 (s), 5.28 (m), 5.18 (m), 4.98 (s), 4.84 (s), 4.55 (m), 4.46 (m), 4.11 (vs), 4.05 (m), 3.85 (m), 3.82 (m), 3.72 (vs), 3.66 (vs), 3.50 (m), 3.40 (m), 3.25 (m); most preferably at 10.5 (m), 10.0 (w), 7.6 (s), 6.8 (m), 6.2 (m), 5.97 (s), 5.65 (w), 5.28 (m), 5.18 (m), 4.98 (s), 4.84 (s), 4.55 (m), 4.46 (m), 4.11 (vs), 4.05 (m), 3.85 (m), 3.82 (m), 3.72 (vs), 3.66 (vs), 3.50 (m), 3.45 (w), 3.40 (m), 3.25 (m), 3.13 (w), 2.81 (w).

Another object of the invention is a crystalline 1,4-dioxane solvate of (S)-4-[[3-[2-(dimethylamino)ethyl]-1H-indol-5-yl]methyl]-2-oxazolidinone, herein designated as Form G, which exhibits a characteristic X-ray powder diffraction pattern with characteristic peaks expressed in d-values (Å) and in 2θ as given in Table 7.

TABLE 7
d-spacings and 2θ angles for Form G.

| Angle °2θ | d-spacing (Å) | Qualitative Relative Intensity |
|---|---|---|
| 8.4 | 10.5 | m |
| 8.9 | 10.0 | vw |
| 11.4 | 7.8 | m |
| 12.7 | 7.0 | w |
| 13.4 | 6.6 | w |
| 13.7 | 6.4 | w |
| 14.3 | 6.2 | vw |
| 15.0 | 5.91 | s |
| 15.5 | 5.72 | m |
| 16.9 | 5.26 | s |
| 17.7 | 4.99 | s |
| 18.3 | 4.85 | vs |
| 19.5 | 4.54 | m |
| 19.8 | 4.47 | m |
| 21.0 | 4.22 | m |
| 21.8 | 4.08 | s |
| 22.2 | 4.00 | w |
| 22.7 | 3.92 | m |
| 22.8 | 3.89 | m |
| 23.2 | 3.82 | m |
| 24.0 | 3.71 | m |
| 24.4 | 3.65 | m |
| 25.6 | 3.48 | m |
| 25.9 | 3.43 | w |
| 27.0 | 3.30 | w |
| 27.5 | 3.24 | w |
| 28.0 | 3.19 | vw |
| 28.5 | 3.12 | w |
| 29.9 | 2.99 | w |
| 31.6 | 2.83 | w |
| 32.0 | 2.80 | vw |
| 32.8 | 2.73 | w |

Besides by the X-ray powder diffraction pattern depicted in FIG. 7, Form G is distinguishably characterised by peaks expressed in d-values (Å) at 5.91 (s), 5.26 (s), 4.99 (s), 4.85 (vs), 4.08 (s); more preferably at 10.5 (m), 7.8 (m), 5.91 (s), 5.72 (m), 5.26 (s), 4.99 (s), 4.85 (vs), 4.54 (m), 4.47 (m), 4.22 (m), 4.08 (s), 3.92 (m), 3.89 (m), 3.82 (m), 3.71 (m), 3.65 (m), 3.48 (m); most preferably at 10.5 (m), 7.8 (m), 7.0 (w), 6.6 (w), 6.4 (w), 5.91 (s), 5.72 (m), 5.26 (s), 4.99 (s), 4.85 (vs), 4.54 (m), 4.47 (m), 4.22 (m), 4.08 (s), 4.00 (w), 3.92 (m), 3.89 (m), 3.82 (m), 3.71 (m), 3.65 (m), 3.48 (m), 3.43 (w), 3.30 (w), 3.24 (w), 3.12 (w), 2.99 (w), 2.83 (w), 2.73 (w).

The polymorphic Form A of Zolmitriptan is especially characterized by an X-ray powder diffraction pattern as depicted in FIG. 1, whereas the Form B is especially characterized by an X-ray powder diffraction pattern as depicted in FIG. 2, the Form C by an X-ray powder diffraction pattern as depicted in FIG. 3, the Form D by an X-ray powder diffraction pattern as depicted in FIG. 4, the Form E by an X-ray powder diffraction pattern as depicted in FIG. 5, the Form F by an X-ray powder diffraction pattern as depicted in FIG. 6, and the Form G by an X-ray powder diffraction pattern as depicted in FIG. 7.

Furthermore, the present invention is directed to processes for the preparation of Form A, B, C, D, E, F and G of Zolmitriptan.

Form A can be generally prepared by crystallization from solutions of Zolmitriptan. Crystallization is conveniently initialized by cooling. Solutions may be formed in pure solvents or mixtures of solvents with non-solvents. For obtaining the desired form A in pure form, solutions of Zolmitriptan used preferably do not contain a solvent or non-solvent forming a solvate crystal (e.g. 1-butanol, anisole, ethyl methyl ketone, tetrahydrofuran, 1,4-dioxane, ethyl acetate), such as forms B, C, E, F and G of Zolmitriptan, whose preparation is described further below. Examples of preferred solvents are lower alcohols (except 2-propanol and 1-butanol), e.g. methanol, ethanol, 1-propanol, 2-butanol, tert.-butanol, or suitable sulfoxides or amides such as dimethylsulfoxide, dimethylformamide. Examples of non-solvents are alkanes or ethers, e.g. C₅-C₈alkanes and/or (non-cyclic) dialkylethers such as diethyl ether, dimethyl ether, methyl-propyl ether, tert.-butyl-methyl ether, preferably diethyl ether, dimethyl ether, tert.-butyl-methyl ether, hexane heptane. For example, form A may be obtained from cooled alcoholic solutions of Zolmitriptan. Preferably, the alcohol used is ethanol or methanol, especially ethanol, and most preferably the alcoholic solutions are mixed with water. Form A can also conveniently be prepared by crystallization from cooled solutions of Zolmitriptan in a mixture of an alcohol as mentioned above with a non-solvent. Most preferably, the alcohol is methanol or ethanol and the non-solvent is an ether.

Preferably crystallization from solution is achieved in that these solutions are cooled from temperatures of about 20 to 100° C. down to temperatures of about −20° to 10°. Most preferably from temperatures of about 50 to 80° C. down to temperatures of about 0° C. to 5° C.

Form A can also be generally prepared by stirring the amorphous form in an organic solvent or non-solvent as described above. The amorphous form is generally obtained as an oil by evaporation of a solution of Zolmitriptan, e.g. an alcoholic solution of Zolmitriptan. Preferably the amorphous form is stirred in a non-solvent, e.g. an ether, most preferably in diethyl ether or methyl tert-butyl ether.

Form A can also be made by dispersing any form of Zolmitriptan, e.g. crystalline or the above amorphous form, in an organic solvent, e.g. those described above. Form A may generally be prepared by suspending Zolmitriptan in an organic solvent. These suspensions generally can be made using any crystalline form of Zolmitriptan. Alternatively, dispersions of amorphous Zolmitriptan may be used. Preferably these dispersions or, preferably, supensions are made in an alcohol or an acetate. Most preferably, the suspension is in 2-propanol and/or ethyl acetate and stirred for several hours and the recovered solid is dried.

Form B can be generally prepared by crystallization from a solution of Zolmitriptan in 1-butanol, or mixtures of 1-butanol with a co-solvent (e.g. an organic solvent as noted above), preferably by evaporation of a solution of Zolmitriptan in 1-butanol, or mixtures of 1-butanol with a co-solvent (e.g. an organic solvent as noted above). Preferably the cosolvent is another alcohol, and the evaporation is performed at atmospheric pressure, e.g. by treating the product with a stream of gas. Most preferably, 1-butanol is the only solvent, or methanol and/or ethanol is the other organic solvent.

Usually, Zolmitriptan form B thus obtained contains up to 20% of 1-butanol, e.g. between about 5 and 20%, especially 8-18% 1-butanol.

Form C can be generally prepared by stirring a suspension of Zolmitriptan in anisole. Usually, Zolmitriptan form C thus obtained contains up to 25% anisole, e.g. between about 10 and 25%, especially 15-25% anisole.

Form D can be generally prepared by crystallization from a 2-propanol solution, usually followed by gentle drying, e.g. at room temperature and atmospheric pressure, advantageously under a stream of gas. Preferably, preparation is done by gentle evaporation of a 2-propanol solution. Most preferably the evaporation is performed by treating the product with a stream of gas, e.g. at atmospheric pressure. Usually, Zolmitriptan form D thus obtained contains up to 20% of 2-propanol, e.g. between about 5 and 20 %, especially 8-18% 2-propanol.

Form E can be generally prepared by crystallization from solutions of Zolmitriptan in ethyl methyl ketone, or by dispersing, preferably suspending, Zolmitriptan in ethyl methyl ketone. Crystallization is conveniently initialized by cooling as described above for form A, and/or by evaporation of the solvent. Preferably the suspension in ethyl methyl ketone is stirred. The product may conveniently be isolated by filtration. Usually, Zolmitriptan form E thus obtained contains up to 15% of ethyl methyl ketone, e.g. between about 5 and 15%.

Form F can be generally prepared by crystallization from a tetrahydrofuran solution, e.g. by cooling as described above for form A. Preferably by gentle evaporation of a tetrahydrofuran solution. Most preferably, the evaporation is performed by treating the product with a stream of gas, e.g. at atmospheric pressure. Usually, Zolmitriptan form F thus obtained contains up to 25% tetrahydrofuran, e.g. between about 10 and 25%, especially 15-25% tetrahydrofuran.

Form G can be generally prepared by crystallization from solutions of Zolmitriptan in 1,4-dioxane, or by dispersing, preferably suspending, Zolmitriptan in 1,4-dioxane. Crystallization is conveniently initialized by cooling as described above for form A, and/or by evaporation of the solvent. Preferably the suspension in 1,4-dioxane is stirred. The product may conveniently be isolated by filtration. Usually, Zolmitriptan form G thus obtained contains up to 25% of 1,4-dioxane, e.g. between about 10 and 25 %, especially 15-25% of 1,4-dioxane.

Where crystalline forms are obtained as a suspension, the product may conveniently be isolated by filtration and/or drying. The suspenstons usually are treated in the temperature range 0-60° C., especially 5-30° C. Where a gas stream is used for the evaporation of solvents and/or non-solvents, the gas employed may be air or an inert gas, e.g. dried air or nitrogen.

In the above mentioned processes small amounts of seeding crystals of the desired crystalline form may be added to the reaction mixture. Preferably small amounts are about 1 to 20 weight %, more preferably about 5 weight % (e.g. 2-10%). Seeding crystals may be added before or, where appropriate, after the step initiating the crystallization (e.g. cooling, addition of non-solvent, evaporation etc. as described above). Addition before initiating the crystallization is of specific technical interest.

Solutions or dispersions of Zolmitriptan used in the above processes may be prepared in situ, e.g. by an Eschweiler-Clark methylation of the free amine.

Another object of the present invention are pharmaceutical compositions comprising an effective amount of crystalline Form A, B, C, D, E, F or G of Zolmitriptan, and a pharmaceutically acceptable carrier.

These polymorphic forms may be used as single component or as mixtures with other crystalline forms or the amorphous form of Zolmitriptan.

As to Zolmitriptan it is preferred that it contains 25-100% by weight, especially 50-100% by weight of a novel form, based on the total amount of Zolmitriptan. Preferably, such an amount of the novel polymorphic form of Zolmitriptan is 75-100% by weight, especially 90-100% by weight. Highly preferred is an amount of 95-100% by weight.

Present invention includes a process for the preparation of a pharmaceutical composition, which process comprises addition of an effective amount of the pharmaceutically active ingredient to a pharmaceutically acceptable carrier. Present invention further includes a pharmaceutical composition comprising an effective amount of the pharmaceutically active ingredient and a pharmaceutically acceptable carrier. Present invention further pertains to the use of this pharmaceutical composition for the manufacturing of a drug intended for the treatment and/or prevention of migraine, or for the manufacturing of a medicament for the treatment and/or prevention of clinical conditions for which a selective antagonist of 5-HT_{1B/1D-}like receptors is indicated. Thus, present invention also includes a method for the treatment and/or prevention of clinical conditions for which a selective antagonist of 5-HT_{1B/1D-}like receptors is indicated, comprising administering to a patient in need of such treatment an effective amount of the pharmaceutical composition of the invention.

The compositions of the present invention include powders, granulates, aggregates and other solid compositions comprising the novel polymorphic form of Zolmitriptan. In addition, the compositions that are contemplated by the present invention may further include diluents, such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents like calcium carbonate and calcium diphosphate and other diluents known to the pharmaceutical industry. Yet other suitable diluents include waxes, sugars and sugar alcohols like mannitol and sorbitol, acrylate polymers and copolymers, as well as pectin, dextrin and gelatin.

Further excipients that are within the contemplation of the present invention include binders, such as acacia gum, pregelatinized starch, sodium alginate, glucose and other binders used in wet and dry granulation and direct compression tableting processes. Excipients that also may be present in the solid compositions further include disintegrants like sodium starch glycolate, crospovidone, low-substituted hydroxypropyl cellulose and others. In addition, excipients may include tableting lubricants like magnesium and calcium stearate and sodium stearyl fumarate; flavorings; sweeteners; preservatives; pharmaceutically acceptable dyes and glidants such as silicon dioxide.

The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well-known in the art of pharmacy.

Dosage forms include solid dosage forms, like tablets, powders, capsules, suppositories, sachets, troches and losenges as well as liquid suspensions and elixirs. While the description is not intended to be limiting, the invention is also not intended to pertain to true solutions of Zolmitriptan whereupon the properties that distinguish the solid form of Zolmitriptan are lost. However, the use of the novel form to prepare such solutions is considered to be within the contemplation of the invention.

Capsule dosages, of course, will contain the solid composition within a capsule which may be made of gelatin or other conventional encapsulating material. Tablets and powders may be coated. Tablets and powders may be coated with an enteric coating. The enteric coated powder forms may have coatings comprising phthalic acid cellulose acetate, hydroxypropylmethyl-cellulose phthalate, polyvinyl alcohol phthalate, carboxymethylethyl§cellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric coating.

Preferred unit dosages of the pharmaceutical compositions of this invention typically contain from 1 to 50 mg of the novel Zolmitriptan forms or mixtures thereof with each other or other forms of Zolmitriptan. More usually, the combined weight of the Zolmitriptan forms of a unit dosage are from 0.5 mg to 30 mg, for example 1, 2.5 or 5 mg.

The following Examples illustrate the invention in more detail. Temperatures are given in degrees Celsius. If not stated otherwise, whereever mentioned, ambient atmosphere or room temperature (RT) is in the range 20-25° C., and percentages are given by weight. Whereever mentioned, atmospheric pressure stands for a pressure of about 105 Pa (e.g. 0.5-about 1.5 bar, especially about 0.9-about 1.1 bar, under conditions commonly used in laboratory or industrial synthesis). Abbreviations and symbols used in the Examples and elsewhere:
TBME: tert.-butyl-methyl ether
1 Å stands for 10⁻¹⁰ m.

### EXAMPLE 1

### Preparation of Form A

83 mg of Zolmitriptan were dissolved in a mixture of 1 ml ethanol and 1 ml water at 60° C. Then slowly 4 ml of water were added after which the slightly turbid solution was cooled to 5° C. at a cooling rate of about 1° C. per minute. The formed suspension was stirred at this temperature for 16 hours and the solid was isolated by filtration and dried at 40° C. for 2 hours. The obtained crystal Form A is characterized by an X-ray powder diffraction pattern as shown in FIG. 1.

### EXAMPLE 2

### Preparation of Form A

81 mg of Zolmitriptan were dissolved in 0.3 ml methanol. Then slowly 3 ml of methyl tert.-butyl ether were added after which the slightly turbid solution was cooled to 5° C. The formed suspension was stirred at this temperature for 16 hours and the solid was isolated by filtration and dried at 40° C. for 2 hours. The obtained crystal Form A is characterized by an X-ray powder diffraction pattern as shown in FIG. 1.

### EXAMPLE 3

### Preparation of Form A

101 mg of Zolmitriptan were dissolved in 1 ml 2-propanol at 60° C. This solution was cooled to 5° C. at a cooling rate of about 1° C. per minute. The formed suspension was stirred at this temperature for 16 hours and the solid was isolated by filtration and dried at 40° C. for 2 hours. The obtained crystal Form A is characterized by an X-ray powder diffraction pattern as shown in FIG. 1.

### EXAMPLE 4

### Preparation of Form A

180 mg of Zolmitriptan were dissolved in 1 ml methanol. This solution was evaporated to dryness in vacuum giving the amorphous Zolmitriptan as an oil. This amorphous material was suspended in 2 ml methyl tert-butyl ether and stirred at room temperature for 4 hours. The crystalline solid was isolated by filtration and dried. The obtained crystal Form A is characterized by an X-ray powder diffraction pattern as shown in FIG. 1.

### EXAMPLE 5

### Preparation of Form A

110 mg Zolmitriptan was suspended in 1 ml 2-propanol and this suspension was stirred at room temperature for 16 hours. The crystalline solid was isolated by filtration and dried at room temperature under vacuum. The obtained crystal Form A is characterized by an X-ray powder diffraction pattern as shown in FIG. 1.

### EXAMPLE 6

### Preparation of Form A

105 mg Zolmitriptan was suspended in 2 ml ethyl acetate and this suspension was stirred at room temperature for 40 hours. The crystalline solid was isolated by filtration and dried at room temperature. The obtained crystal Form A is characterized by an X-ray powder diffraction pattern as shown in FIG. 1.

### EXAMPLE 7

### Preparation of Form B

70 mg Zolmitriptan was dissolved in a mixture of 1 ml 1-butanol and 1 ml water free ethanol. This solution was evaporated to dryness under a weak flow of dry nitrogen over a period of 16 hours. The obtained crystal Form B is characterized by an X-ray powder diffraction pattern as shown in FIG. 2. A TG-FTIR analysis showed that the product contains about 10% 1-butanol.

### EXAMPLE 8

### Preparation of Form B

49 mg Zolmitriptan was dissolved in 2 ml 1-butanol at room temperature. This solution was evaporated to dryness under a weak flow of dry nitrogen. The obtained crystal Form B is characterized by an X-ray powder diffraction pattern as shown in FIG. 2. A TG-FTIR analysis showed that the product contains about 16% 1-butanol.

### EXAMPLE 9

### Preparation of Form C

72 mg Zolmitriptan were suspended in 1 ml anisole, and this suspension was stirred at room temperature for 18 hours. The crystalline solid was isolated by filtration and dried at room temperature. The obtained crystal Form C is characterized by an X-ray powder diffraction pattern as shown in FIG. 3. A TG-FTIR analysis showed that the product contains about 23% anisole.

### EXAMPLE 10

### Preparation of Form D

110 mg Zolmitriptan were dissolved in 4 ml 2-propanol at 60° C. After cooling the solution to room temperature the solvent is evaporated using a stream of dry nitrogen gas. The obtained crystalline Form D is characterized by an X-ray powder diffraction pattern as shown in FIG. 4. A TG-FTIR analysis showed that the product contains about 13% 2-propanol.

### EXAMPLE 11

### Preparation of Form E

75 mg Zolmitriptan are suspended in 1 ml ethyl methyl ketone, and stirred at room temperature for 16 hours. The crystalline solid was isolated by filtration and dried at room temperature. The obtained crystal Form E is characterized by an X-ray powder diffraction pattern as shown in FIG. 5. A TG-FTIR analysis showed that the product contains about 11% ethyl methyl ketone.

### EXAMPLE 12

### Preparation of Form F

50 mg Zolmitriptan are dissolved in 2 ml tetrahydrofuran. This solution was evaporated to dryness using a stream of dry nitrogen gas. The obtained crystal Form F is characterized by an X-ray powder diffraction pattern as shown in FIG. 6. A TG-FTIR analysis showed that the product contains about 17% tetrahydrofuran.

### EXAMPLE 13

### Preparation of Form G

79 mg Zolmitriptan are suspended in 1 ml 1,4-dioxane, and stirred at room temperature for about 16 hours. The crystalline solid was isolated by filtration and dried at room temperature. The obtained crystal Form G is characterized by an X-ray powder diffraction pattern as shown in FIG. 7. A TG-FTIR analysis showed that the product contains about 21% 1,4-dioxane.

Powder X-ray Diffraction Analysis

PXRD was performed on a Philips 1710 powder X-ray diffractometer using Cu_{Kα} radiation. D-spacings were calculated from the 20 using the wavelength of the Cu_{Kα1} radiation of 1.54060 A. The X-ray tube was operated at a Voltage of 45 kV, and a current of 45 mA. A step size of 0.02°, and a counting time of 2.4 s per step was applied. Generally, 2θ values are within an error of ±0.1-0.2°. The experimental error on the d-spacing values is therefore dependent on the peak location.

### BRIEF DESCRIPTION OF THE DRAWINGS

FIG. 1 is a characteristic X-ray powder diffraction pattern for Form A

FIG. 2 is a characteristic X-ray powder diffraction pattern for Form B

FIG. 3 is a characteristic X-ray powder diffraction pattern for Form C

FIG. 4 is a characteristic X-ray powder diffraction pattern for Form D

FIG. 5 is a characteristic X-ray powder diffraction pattern for Form E

FIG. 6 is a characteristic X-ray powder diffraction pattern for Form F

FIG. 7 is a characteristic X-ray powder diffraction pattern for Form G

1. A crystalline polymorph A of (S)-4-[[3-[2-(dimethylamino)ethyl]-1 H-indol-5-yl]methyl]-2-oxazolidinone which exhibits a characteristic X-ray powder diffraction pattern with characteristic peaks expressed in d-values (Å) at 6.4 (s), 6.15 (s), 5.69 (s), 4.59 (vs), 4.53 (s), 4.02 (s), 3.71 (vs), 3.08 (s); wherein (vs)=very strong intensity; (s)=strong intensity.

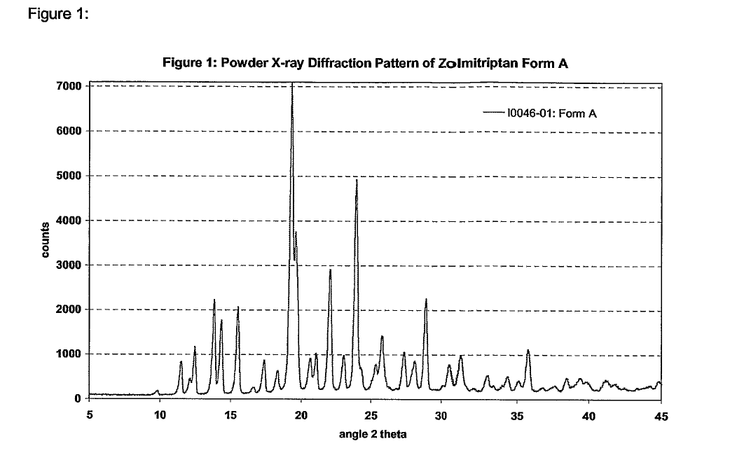

2. A crystalline polymorph A of (S)-4-[[3-[2-(dimethylamino)ethyl]-1H-indol-5-yl]methyl]-2-oxazolidinone according to claim 1, having an X-ray powder diffraction pattern substantially as depicted in FIG. 1.

3. A crystalline 1-butanol solvate of (S)-4-[[3-[2-(dimethylamino)ethyl]-1H-indol-5-yl]methyl]-2-oxazolidinone which exhibits a characteristic X-ray powder diffraction pattern with characteristic peaks expressed in d-values (Å) at 7.5 (s), 4.87 (s), 4.48 (s), 4.05 (s), 3.76 (s); wherein (s)=strong intensity.

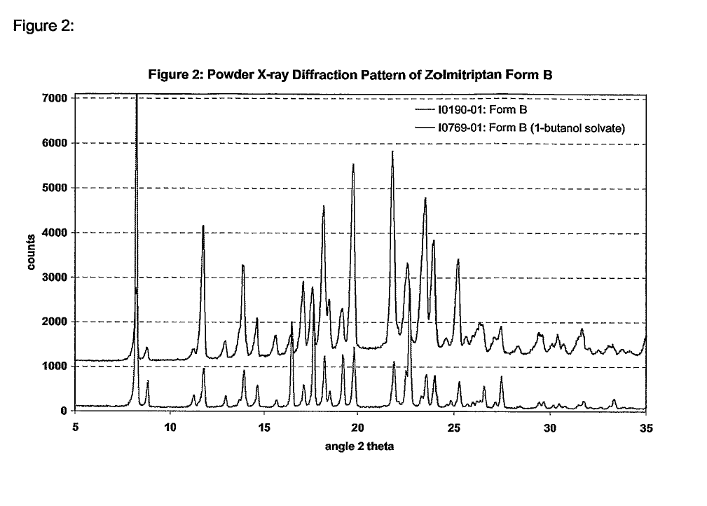

4. A crystalline 1-butanol solvate of (S)-4-[[3-[2-(dimethylamino)ethyl]-1H-indol-5-yl]methyl]-2-oxazolidinone according to claim 3, having an X-ray powder diffraction pattern substantially as depicted in FIG. 2.

5. A crystalline 1-butanol solvate of (S)-4-[[3-[2-(dimethylamino)ethyl]-1H-indol-5-yl]methyl]-2-oxazolidinone according to claim 3 containing up to 20% of 1 butanol, relative to the weight of the crystalline solvate.

6. A crystalline anisol solvate of (S)-4-[[3-[2-(dimethylamino)ethyl]-1H-indol-5-yl]methyl]-2-oxazolidinone which exhibits a characteristic X-ray powder diffraction pattern with characteristic peaks expressed in d-values (Å) at 7.8 (s), 6.4 (s), 4.89 (s), 4.44 (vs), 4.00 (s), 3.70 (vs), 3.46 (s); wherein (vs)=very strong intensity; (s)=strong intensity.

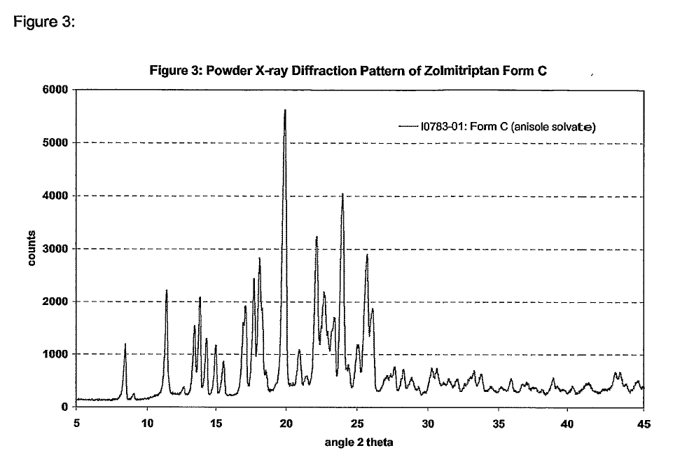

7. A crystalline anisol solvate of (S)-4-[[3-[2-(dimethylamino)ethyl]-1H-indol-5-yl]methyl]-2-oxazolidinone according to claim 6, having an X-ray powder diffraction pattern substantially as depicted in FIG. 3.

8. A crystalline anisol solvate of (S)-4-[[3-[2-(dimethylamino)ethyl]-1H-indol-5-yl]methyl]-2-oxazolidinone according to claim 6 containing up to 25% anisole.

9. A crystalline isopropanol solvate of (S)-4-[[3-[2-(dimethylamino)ethyl]-1H-indol-5-yl]methyl]-2-oxazolidinone which exhibits a characteristic X-ray powder diffraction pattern with characteristic peaks expressed in d-values (Å) at 10.7 (s), 7.6 (vs), 6.3 (s), 5.21 (s), 5.03 (s), 4.86 (vs), 4.50 (vs), 4.11 (s), 3.90 (s), 3.69 (s), 3.52 (s); wherein (vs)=very strong intensity; (s)=strong intensity.

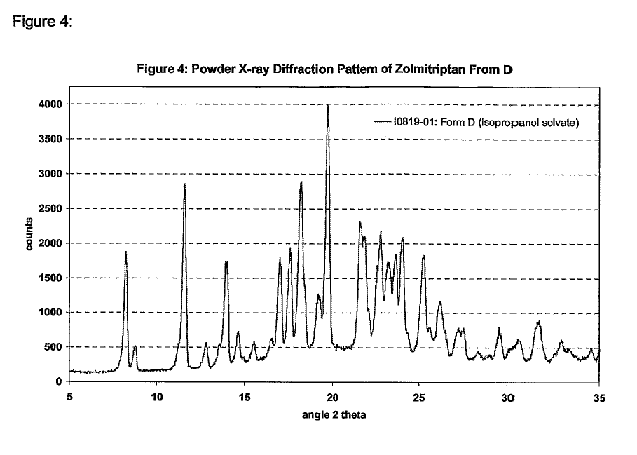

10. A crystalline isopropanol solvate of (S)-4-[[3-[2-(dimethylamino)ethyl]-1H-indol-5-yl]methyl]-2-oxazolidinone according to claim 9, having an X-ray powder diffraction pattern substantially as depicted in FIG. 4.

11. A crystalline isopropanol solvate of (S)-4-[[3-[2-(dimethylamino)ethyl]-1H-indol-5-yl]methyl]-2-oxazolidinone according to claim 9 containing up to 20% isopropanol.

12. A crystalline ethyl methyl ketone solvate of (S)-4-[[3-[2-(dimethylamino)ethyl]-1H-indol-5-yl]methyl]-2-oxazolidinone which exhibits a characteristic X-ray powder diffraction pattern with characteristic peaks expressed in d-values (Å) at 7.3 (vs), 6.2 (s), 4.85 (s), 4.66 (s), 4.47 (vs), 4.03 (s), 3.98 (s), 3.72 (s), 3.55 (s); wherein (vs)=very strong intensity; (s)=strong intensity.

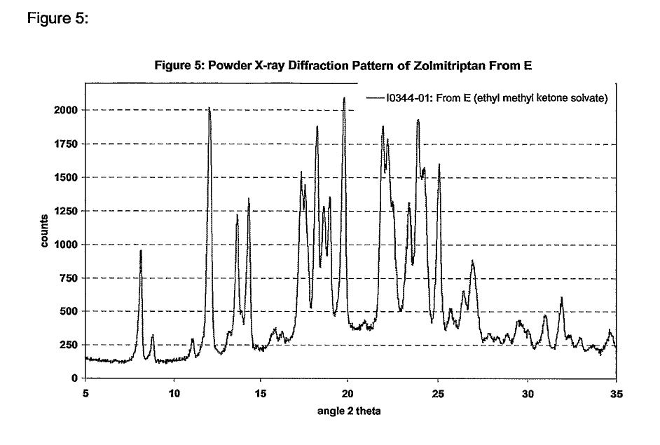

13. A crystalline ethyl methyl ketone solvate of (S)-4-[[3-[2-(dimethylamino)ethyl]-1H-indol-5-yl]methyl]-2-oxazolidinone according to claim 12, having an X-ray powder diffraction pattern substantially as depicted in FIG. 5.

14. A crystalline ethyl methyl ketone solvate of (S)-4-[[3-[2-(dimethylamino)ethyl]-1H-indol-5-yl]methyl]-2-oxazolidinone according to claim 12 containing up to 15% ethyl methyl ketone.

15. A crystalline tetrahydrofuran solvate of (S)-4-[[3-[2-(dimethylamino)ethyl]-1H-indol-5-yl]methyl]-2-oxazolidinone which exhibits a characteristic X-ray powder diffraction pattern with characteristic peaks expressed in d-values (Å) at 7.6 (s), 5.97 (s), 4.98 (s), 4.84 (s), 4.11 (vs), 3.72 (vs), 3.66 (vs); wherein (vs)=very strong intensity; (s)=strong intensity.

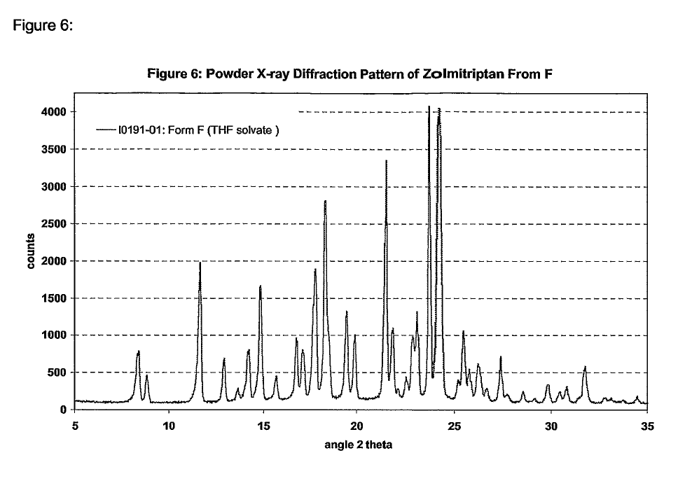

16. A crystalline tetrahydrofuran solvate of (S)-4-[[3-[2-(dimethylamino)ethyl]-1H-indol-5-yl]methyl]-2-oxazolidinone according to claim 15, having an X-ray powder diffraction pattern substantially as depicted in FIG. 6.

17. A crystalline tetrahydrofuran solvate of (S)-4-[[3-[2-(dimethylamino)ethyl]-1H-indol-5-yl]methyl]-2-oxazolidinone according to claim 15 containing up to 25% tetrahydrofuran.

18. A crystalline 1,4-dioxane solvate of (S)-4-[[3-[2-(dimethylamino)ethyl]-1H-indol-5-yl]methyl]-2-oxazolidinone which exhibits a characteristic X-ray powder diffraction pattern with characteristic peaks expressed in d-values (Å) at 5.91 (s), 5.26 (s), 4.99 (s), 4.85 (vs), 4.08 (s); wherein (vs)=very strong intensity; (s)=strong intensity.

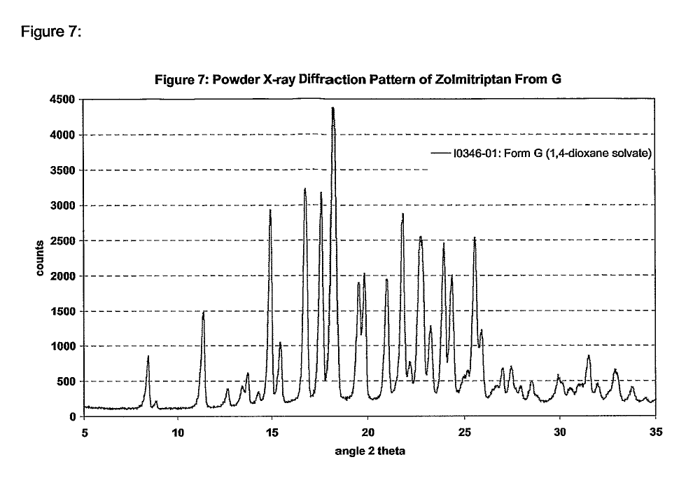

19. A crystalline 1.4-dioxane solvate of (S)-4-[[3-[2-(dimethylamino)ethyl]-1H-indol-5-yl]methyl]-2-oxazolidinone according to claim 18, having an X-ray powder diffraction pattern substantially as depicted in FIG. 7.

20. A crystalline 1.4-dioxane solvate of (S)-4-[[3-[2-(dimethylamino)ethyl]-1H-indol-5-yl]methyl]-2-oxazolidinone according to claim 18 containing up to 25% of 1,4-dioxane.

21. A process for the manufacture of crystalline polymorph A of (S)-4-[[3-[2-(dimethylamino)ethyl]-1H-indol-5-yl]methyl]-2-oxazolidinone according to claim 1 wherein a solution of Zolmitriptan in an organic solvent or mixture of organic solvents is cooled, provided that the solution does not contain 1-butanol, anisole, 2-propanol, ethyl methyl ketone, tetrahydrofuran, 1,4-dioxane, or ethyl acetate.

22. A process according to claim 21, wherein an organic solvent is selected from C₁-C₄alkanols, sulfoxides, and/or amides, or mixtures of C₁-C₄alkanols with water.

23. A process according to claim 21, wherein the solution additionally contains a non-solvent selected from alkanes and ethers.

24. A process according to claim 21 in which the solution is cooled from a temperature of about 20° to 100° C. down to about −20° C. to 10° C.

25. A process for the manufacture of crystalline polymorph A of (S)-4-[[3-[2-(dimethylamino)ethyl]-1H-indol-5-yl]methyl]-2-oxazolidinone according to claim 1 wherein crystalline Zolmitriptan is suspended, or amorphous Zolmitriptan is dispersed, in an organic solvent, provided that the solvent does not contain 1-butanol, anisole, ethyl methyl ketone, tetrahydrofuran, or 1,4-dioxane.

26. A process according to claim 25, wherein the organic solvent is an alcohol or an acetate.

27. A process for the manufacture of crystalline polymorph B of (S)-4-[[3-[2-(dimethylamino)ethyl]-1H-indol-5-yl]methyl]-2-oxazolidinone according to claim 3, by cooling, or solvent evaporation, of a solution of Zolmitriptan in 1-butanol or in a solvent containing 1-butanol, provided that the solvent does not contain anisole, ethyl methyl ketone, 2-propanol, tetrahydrofuran, 1,4-dioxane, or ethyl acetate.

28. A process for the manufacture of crystalline polymorph C of (S)-4-[[3-[2-(dimethylamino)ethyl]-1H-indol-5-yl]methyl]-2-oxazolidinone according to claim 6, wherein a suspension of Zolmitriptan is stirred in anisole.

29. A process for the manufacture of crystalline polymorph D of (S)-4-[[3-[2-(dimethylamino)ethyl]-1H-indol-5-yl]methyl]-2-oxazolidinone according to claim 9, wherein a solution of Zolmitriptan in 2-propanol is cooled and/or the 2-propanol is evaporated.

30. A process for the manufacture of crystalline polymorph E of (S)-4-[[3-[2-(dimethylamino)ethyl]-1H-indol-5-yl]methyl]-2-oxazolidinone according to claim 12, wherein a dispersion of Zolmitriptan is stirred in ethyl methyl ketone, or wherein a solution of Zolmitriptan in ethyl methyl ketone or in a solvent containing ethyl methyl ketone, provided that the solvent does not contain 1-butanol, anisole, 2-propanol, tetrahydrofuran, 1,4-dioxane, or ethyl acetate, is subjected to cooling and/or solvent evaporation.

31. A process for the manufacture of crystalline polymorph F of (S)-4-[[3-[2-(dimethylamino)ethyl]-1H-indol-5-yl]methyl]-2-oxazolidinone according to claim 15, wherein a solution of Zolmitriptan in tetrahydrofuran is cooled and/or the tetrahydrofuran is evaporated.

32. A process for the manufacture of crystalline polymorph G of (S)-4-[[3-[2-(dimethylamino)ethyl]-1H-indol-5-yl]methyl]-2-oxazolidinone according to claim 18, wherein Zolmitriptan is suspended in 1,4-dioxane, or wherein a solution of Zolmitriptan in 1,4-dioxane or in a solvent containing 1,4-dioxane, provided that the solvent does not contain 1-butanol, anisole, 2-propanol, methyl ethyl ketone, tetrahydrofuran, or ethyl acetate, is subjected to cooling and/or solvent evaporation.

33. A process according to claim 21, wherein seeding is carried out with crystals of the desired crystalline polymorph.

34. A process according to claim 21 in which the solution or dispersion of Zolmitriptan is prepared in situ.

35. A pharmaceutical composition comprising a crystalline polymorphic form according to claim 1, and a pharmaceutically acceptable carrier.

36. Zolmitriptan containing a crystalline polymorphic form according to claim 1.

37. (canceled)

38. A method for the treatment and/or prevention of clinical conditions for which a selective antagonist of 5-HT_{1B/1D-} like receptors is indicated, comprising administering to a patient in need of such treatment an effective amount of the pharmaceutical composition according to claim 35.
